(19) United States
(12) Patent Application Publication
(10) Pub. No.: US 2026/0283953 A1
(43) Pub. Date: Sep. 24, 2026

(54) FORMULATIONS OF PHOSPHOLIPID CARRIER COMPLEXES

(71) Applicants: Joseph Halstead, Singapore (SG); Oliver J. Ratcliffe, Alameda, CA (US)

(72) Inventors: Joseph Halstead, Singapore (SG); Oliver J. Ratcliffe, Alameda, CA (US)

(21) Appl. No.: 19/646,278

(22) Filed: Apr. 13, 2026

Related U.S. Application Data
(63) Continuation-in-part of application No. PCT/US2025/017361, filed on Feb. 26, 2025.
(60) Provisional application No. 63/559,814, filed on Feb. 29, 2024.

(51) Int. Cl. A61K 9/127; A61K 31/12; A61K 31/565; A61K 31/568; A61K 31/57; A61P 29/00

(52) U.S. Cl. CPC A61K 9/127; A61K 31/12; A61K 31/565; A61K 31/568; A61K 31/57; A61P 29/00

(57) ABSTRACT

The present disclosure provides formulations for biologically active molecules including steroid hormones, diarylheptanoids, vitamins and flavonoids. These formulations are distinguished from the prior art through the inclusion of magnesium stearate or a salt with comparable properties along with a phospholipid carrier complex. These formulations provide for more convenient modes of delivery, such as via orally consumed capsules containing a solid powder. The present disclosure also provides for the use of combinations of biologically active molecules flavonoids and/or steroid hormone, including the combined delivery of the hormone testosterone and curcuminoid compounds to human and/or animal subjects.

### CROSS-REFERENCES TO RELATED APPLICATIONS

This application claims benefit and is a continuation in part of PCT Application No. PCT/US2025/017361, filed Feb. 26, 2025, which claims priority to U.S. Patent Application No. 63/559,814, filed Feb. 29, 2024, the entire contents of which are incorporated herein by reference for all purposes.

### FIELD OF THE INVENTION

The invention relates to formulations of phospholipid carrier complexes and uses thereof to enable unique modes of delivery of biologically active molecules such as steroid hormones and flavonoids.

### BACKGROUND

Steroid hormones, diarylheptanoids, and/or flavonoids are commonly used as therapeutics, taken either as nutritional supplements or prescription drugs, to address a wide range of health problems. These include the application of testosterone to address weight gain, low libido, and low energy and to reduce the appearance of female secondary sexual features in transgender males.

Curcumin is typically consumed in the diet and is considered to have potential as an anti-inflammatory agent to protect the human body against the effects of inflammation, which has been associated with a range of maladies including of cancer, cardiovascular disease, respiratory disease, age-related diseases, and neurological disorders. However, the use of these molecule classes has been limited by suboptimal formulations which restrict their modes of delivery. Notably, curcumin has been considered to have low potential as a therapeutic drug due to its low bioavailability and instability (see Nelson et al. (2017), J. Med. Chem. 60: 1620-1637, and Nelson et al hitherto. (2017), Med. Chem. Letters. 8: 467-470).

A hallmark of healthy young adult human males is a marked diurnal cycling of testosterone; peak levels occur in a morning and fall to a minimum in the evening. By contrast in elderly males, it is difficult to discern a daily cycle of testosterone levels, and peak levels are significantly lower than the lowest levels in young adult males (Brenner et al. (1983), J. Clin. Endocrinol. Metab., 56. 1278-1281; doi.org/10.1210/jcem-56-6-1278). A key challenge with existing testosterone replacement therapy is in replicating this natural diurnal cycle. Reports on the testosterone market, from as recently as 2021 indicate that there are no significant oral formulations in the market, and the market is divided roughly 50:50 between injectable and topical formulations (see: www. skyquestt. com/report/testosterone-replacement-therapy-market). There has been an approval for oral testosterone undecanoate in the US, but it should be noted that this comprises the ester form of the molecule. Undecanoate is a long-acting ester (half-life 21 days); the principle of the inventions disclosed herein is that the formulations incorporate natural testosterone, which has a half-life of under 1 hour. An undesirable feature of the testosterone esters is that the long half-life does not replicate the natural diurnal cycle of testosterone, which can lead to long-term negative side effects.

The following commentary has been published on Wikipedia: testosterone is documented to be well-absorbed but extensively metabolized with oral deliver due to the first pass through the intestines and liver. It is rapidly and completely inactivated in men at doses of less than 200 mg. In large doses, such as 200 mg, however, significant increases in circulating testosterone levels become apparent. In addition, while a 60 mg dose has no effect on testosterone levels in men, this dose does measurably increase testosterone levels in prepubertal boys and women (Wikipedia). The oral bioavailability of testosterone in young women after a single 25 mg dose was found to be 3.6±2.5%. High levels of testosterone are also achieved with a 60 mg dose of oral testosterone in men with liver cirrhosis..

The above findings have been attributed to induction of liver enzymes by testosterone and consequent activation of its own metabolism. Substitution dosages of oral testosterone in men are in the range of 400 to 800 mg/day. Such doses exceed the amount of testosterone produced by the body, which is approximately 7 mg/day, by approximately 100-fold. The elimination half-life of oral testosterone is rapid at about 5 to 7 hours. As a result, it requires administration several times per day in divided doses.

Due to its limitations, such as the high doses required and necessity of multiple daily doses, oral testosterone is not used clinically in its unmodified form. Herein we disclose formulations for steroid hormone and flavonoid active ingredients, which provide for more convenient oral delivery and a greater flexibility in delivery to patients. Specifically, we disclose a solid liposomal formulation of testosterone which can be taken orally, and which allows for slower absorption as well as, to some extent, bypassing of the liver. If taken at night and early morning by older men, it is expected that this solid liposomal formulation will allow for slower absorption as well as, to some extent, bypassing of the liver, to deliver testosterone levels that approximate the natural levels found in younger men.

We further disclose formulations comprising beneficial combinations of steroid and flavonoid active ingredients.

### SUMMARY

The disclosed formulations, methods and processes can be applied for use to produced formulated health supplement products or prescription drugs. Specifically, we disclose the use of magnesium stearate and/or a salt with comparable properties as a component within formulations comprising a flavonoid molecule and/or a steroid hormone and a phospholipid carrier complex. The inclusion of magnesium stearate or a comparable salt offers important improvements over the existing art and enables the delivery of these active molecules in solid form (for example in tablet or capsule), such that they can be delivered orally. A particular example comprises a formulation containing testosterone, a phospholipid carrier complex, and magnesium stearate, provided as capsule based oral formulation. This method of delivery is superior to the existing modes of delivery, which typically involve application via skin patches or by injection. We further disclose combination formulations, including both testosterone and curcumin, wherein the latter is included to protect against inflammation and/or cardiovascular disease which might otherwise result from increased testosterone levels.

As used herein, the terms “comprising,” “containing,” and “including” are inclusive, open-ended and do not exclude additional unrecited elements, compositional components or method steps. Accordingly, the terms “comprising” and “including” encompass the more restrictive terms “consisting of” and “consisting essentially of.”

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

As used herein, the terms “a,” “an,” and “the” include plural reference unless the context dictates otherwise. For example, if the a,” “an,” and “the” include plural reference unless the context dictates otherwise.

### SPECIFIC EMBODIMENTS

Liposomes are closed bilayer structures spontaneously formed by hydrated phospholipids that are widely used as efficient delivery systems for drugs or antigens, due to their capability to encapsulate bioactive hydrophilic, amphipathic, and lipophilic molecules into inner water phase or within lipid leaflets (Nisini et al., 2018, Front. Immunol., Sec. Vaccines Molec. Therapeutics 9: 155; also reviewed by Liu et al. (2022). Molecules. 27: 1372). Phospholipid carrier complexes are a class of liposomal product which have various applications in drug formulation including to enhance bioavailability of drugs with low aqueous solubility or low membrane penetration, improvement of the uptake and release profile of drugs, protection of sensitive bioactive agents from degradation in the GIT tract, reduction of side effects and masking of bitter taste of drugs. Phospholipids have the exceptional biocompatibility and remarkable amphiphilicity characteristics that make them suitable agents to achieve better therapeutic applications in many drug-delivery systems (Singh et al. (2017), J. Drug Delivery Sci. Technol. 39: 166-179).

Phospholipid carrier complexes have been used with various bioactive chemicals for many years, as have other methods of making liposomes. These liposomal products can be divided into two types of formulation: the very simple, typically based on 1-3 ingredients which mostly occur in nature, versus the very complex which contain purely synthetic components (for example mRNA-based vaccines). Typically, the simple methods of formulation involve the inclusion of lecithin or phosphatidylcholine (one of the main components of lecithin) or bile salts as the bulk of the liposomal carrier. The problems with these simple formulation methods are that (a) if used in powder form, lecithin and phosphatidylcholine are hygroscopic and quickly become very sticky and unusable, and (b) if used in liquid form, a substantial quantity of antimicrobial preservative is required to prevent rot and oxidation of the bioactive ingredient. In the latter applications, since the liposomal formulation acts to make molecules more bioavailable, the preservative compounds also become more bioavailable, which is undesirable and can cause side effects in the subject.

The formulation method disclosed herein involves the addition of a significant quantity (over 10-20% by weight) of magnesium stearate (or a salt with comparable properties) as a hydrophobic component, to prevent the absorption of water from the air or surrounding environment during storage. The inclusion of a hydrophobic component like magnesium stearate in such formulations prevents them from becoming sticky and maintains them in a usable powder form.

Additionally, the addition of magnesium stearate or comparable agent into the formulation may facilitate the rapid dispersion of the phosphatidylcholine/chemical complex once it enters an aqueous environment (in the intestines) leading to more rapid absorption of the liposomal complex and thereby achieving an increased efficacy of the bioactive ingredient in the human or animal subject.

In another series of embodiments, the methodologies herein encompass uses and delivery methods of curcumin as solid formulations (aka. dry formulations). Curcumin and related compounds have previously been delivered in injectable formulations as detailed in: patents.google.com/patent/AU2020204501B2/en.

In a further series of embodiments, the methodologies herein encompass uses of protective molecules of the flavonoid class. Such molecules include luteolin, kaempferol, apigenin and quercetin and derivatives thereof; the aforementioned four common flavanol glycoside compounds are found in many plants and possess multiple biological activities, including anti-inflammatory and antioxidant activities (Tian et al. (2021) South African J. Bot. 137: 257-264).

### Definitions

“ADHD” means “Attention Deficit Hyperactivity Disorder”. ADHD is a common neurodevelopmental disorder which is often first diagnosed in childhood and often lasts into adulthood. Persons with ADHD may have trouble paying attention, concentrating, controlling impulsive behaviors (may act without thinking about what the result will be), or be overly active. Other features of ADHD may include one or more of the following behaviors: daydreaming, forgetfulness, fidgeting, excessive talking, risk taking, irritability and/or aggression.

“Attentional Control” refers to an individual's capacity to choose what they pay attention to and what they ignore and is also colloquially referred to as “Concentration”. Enhanced attentional control is highly desirable for various occupations including pilots, surgeons, and computer gamers.

“AUC” means Area Under Curve, a standard measurement made in pharmacokinetics which indicates the concentration of a drug in the bloodstream versus the time since dosing. In practice, the drug concentration is measured at certain discrete points in time and the trapezoidal rule is used to estimate AUC. In pharmacology, the area under the plot of plasma concentration of a drug versus time after dosage indicates the extent of exposure to a drug and its clearance rate from the body.

“CYP3A4 Inhibitor” means a molecule that has an inhibitory effect on the enzyme Cytochrome P450 3A4 (which is often referred to as “CYP3A4”). CYP3A4 is a member of the cytochrome P450 family of oxidizing enzymes which influence drug metabolism, and which are believed to break down toxins in the liver. Addition of an inhibitor of these enzymes to a formulation may reduce the rate at which a desired drug or bioactive molecule is broken down in the body.

“Curcumin” refers to the bright yellow chemical which is also known as diferuloylmethane. It is produced by plants of the Curcuma longa species and is the principal curcuminoid of turmeric (Curcuma longa), a member of the ginger family, Zingiberaceae. The compound is often included in herbal supplements, cosmetics ingredient, food flavoring, and is sometimes used as a food coloring. Chemically, curcumin is a diarylheptanoid, a molecule that is a polyphenol, a beta-diketone, an enone, a diarylheptanoid and an aromatic ether.

“Dehydroepiandrosterone” or “DHEA” means the Steroid molecule that has been variously referred to as 3β-Hydroxyandrost-5-en-17-one, by its systematic IUPAC name (3aS,3bR,7S,9aR,9bS,11aS)-7-Hydroxy-9a,11a-dimethyl-2,3,3a,3b,4,6,7,8,9,9a,9b,10,11,11a-tetradecahydro-1H-cyclopenta[a]phenanthren-1-one, as androstenolone; as prasterone; as androst-5-en-3β-ol-17-one; as 5,6-Didehydroepiandrosterone; and/or as dehydroisoepiandrosterone. The molecule may act as and/or be used as an androgen.

“Estrogen” means the class of female sex hormones that is responsible for the development and regulation of the female reproductive system and secondary sex characteristics. Several types of estrogen molecule are known to have estrogenic hormonal activity, including estrone, estradiol, estriol and estetrol (which is produced during pregnancy). The former molecules are all analogs with substituents of the same four-ring carbon skeleton or scaffold.

“Flavonoid” means the class of natural product compounds often produced with potential applications in medicinal chemistry. Flavonoids are compounds from the broader class as compounds known as “polyphenols”, Flavonoids (also sometimes referred to as bioflavonoids; from the Latin word flavus, meaning yellow, a common color of such compounds in nature) are a class of polyphenolic secondary metabolites. Chemically, flavonoids have the general structure of a 15-carbon skeleton, which consists of two phenyl rings (A and B) and a heterocyclic ring (C, the ring containing the embedded oxygen). This carbon structure can be abbreviated C6-C3-C6. Flavonoid subtypes include Flavanols, Flavan-3-ols, Flavones, Flavanones, Isoflavones, and Anthocyanins. As noted above, the compounds luteolin, kaempferol, apigenin, and quercetin are examples of flavanols.

“Hormone” means a molecule that typically acts as a signaling molecule in a multicellular organism; such a molecule is typically produced and/or secreted in a particular cell or tissue and them moves to a different cell or tissue where it elicits a physiological or developmental effect. Hormones are organic molecules, and many different classes have been identified. A well-known example is the class comprising Steroid Hormones which includes the sex hormones testosterone, estrogen, and progesterone.

“IBS” means Irritable Bowel Syndrome; a common disorder that affects the gastrointestinal tract. Typical symptoms include cramping, abdominal pain, bloating, gas, diarrhea and/or constipation.

“Inflammation” refers to a response of a human or animal body to harmful stimuli, such as pathogens, damaged cells, or chemical stimuli. The response has evolved as a protective mechanism and involves immune cells, blood vessels, and molecular mediators. Inflammation serves to clear out damaged cells and tissues, and initiate tissue repair. Inflammation can be classified as acute or chronic. Acute inflammation is the initial response of the body to harmful stimuli, and is achieved by the increased movement of plasma and leukocytes (in particular granulocytes) from the blood into the injured tissues (Wikipedia). A series of biochemical events propagates and matures the inflammatory response, involving the local vascular system, the immune system, and various cells in the injured tissue. Prolonged inflammation, known as chronic or systemic inflammation, leads to a progressive shift in the type of cells present at the site of inflammation, such as mononuclear cells, and involves simultaneous destruction and healing of the tissue.

“Inflammatory disease” refers to one or more pathologies that arise due to prolonged inflammation or an overactive inflammation response and may involve the immune system cells attacking the body's own tissues. Drugs, or stimulants such as alcohol, can produce inflammation; chronic inflammation is often associated with aging and general poor health and has been associated with certain cancers, autoimmune diseases, mental health problems, neurodegenerative diseases, and cardiovascular diseases.

“Lecithin” or “lecithins” refer to one or more fatty substance(s) occurring in, or derived from, animal or plant tissues, which shows amphiphilic properties; that is, they attract both water and fatty substances (and thus are both hydrophilic and lipophilic). Lecithins are mixtures that include glycerophospholipids such as phosphatidylcholine, phosphatidylethanolamine, phosphatidylinositol, phosphatidylserine, and/or phosphatidic acid. Lecithin can readily be extracted chemically using organic chemical solvents or extraction can be done mechanically. Common substrates from which lecithin is extracted include egg yolk, seafood, soybeans, milk, rapeseed, cottonseed, and sunflower oil. Lecithin has low solubility in water, but can be included in drug formulations to fulfil the role of a liposomal carrier complex. In aqueous solution, the phospholipids of lecithin can form either liposomes, bilayer sheets, micelles, or lamellar structures, depending on hydration and temperature.

“Lethargy” refers to a condition whereby a human or animal feels, or exhibits symptoms, of fatigue or sluggishness. Lethargy is often associated with changes in mood, reduced mental acuity, decreased alertness, or decreased ability to execute complex tasks.

“Liposome” refers to a self-assembled (phospho)lipid-based drug vesicle that forms a bilayer (unilamellar) and/or a concentric series of multiple bilayers (multilamellar) enclosing a central compartment, which may contain a drug molecule.

“Liposomal carrier complex” or “phospholipid carrier complex” refers to a liposome to which additional chemical components have been added to reduce biodegradation and enhance the efficiency of delivery. Liposomal carrier complexes may incorporate an amphiphilic substance such as lecithin or a polymer, which can increase the stability of liposome with regard to pH, chemicals, enzymes, and the immune system.

“Piperine refers to the molecule with the preferred IUPAC name (2E,4E)-5-(2H-1,3-Benzodioxol-5-yl)-1-(piperidin-1-yl)penta-2,4-dien-1-one. It is sometimes alternatively referred to as Piperoylpiperidine or Bioperine. Piperine, and possibly also its isomer chavicine, is found in black pepper and long pepper and is sometimes included in traditional medicines. The molecule dissolves poorly in water and is sometimes extracted from black pepper using organic solvents such as dichloromethane.

“Polyphenols” refers to the large family of naturally occurring phenols that is abundant in plants and structurally diverse. The term polyphenol is not well-defined, but it is generally agreed that they are natural products that possess one or more hydroxyl groups on aromatic rings and which include four principal classes: phenolic acids, flavonoids, stilbenes, and lignans.

“Progesterone” refers to the steroid molecule that is variously referred to as pregn-4-ene-3,20-dione, (1S,3aS,3bS,9aR,9bS,11aS)-1-Acetyl-9a,11a-dimethyl 1,2,3,3a,3b,4,5,8,9,9a,9b,10,11,11a-tetradecahydro-7H-cyclopenta[a]phenanthren-7-one and or as pregnenedione. The molecule has important endogenous roles in regulating the menstrual cycle, pregnancy, and embryogenesis. It has various medical applications as a drug including uses to mitigate the risk of uterine or cervical cancer, in hormone replacement therapy, in feminizing hormone therapy, and in combination with estrogen as a contraceptive.

“Steroid hormone” refers to a “Steroid” or “Steroid molecule” that acts as a hormone. A “Steroid” or “Steroid molecule” typically means an organic molecule with four fused rings (designated A, B, C, and D) arranged in a specific molecular configuration. Some examples of Steroids are gonane, testosterone, estradiol, progesterone, DHEA, cholic acid, dexamethasone, and lanosterol.

“Testosterone” refers to the Steroid molecule that is the primary male sex hormone and androgen in male animals, with the chemical formula C₁₉H₂₈O₂ and the IUPAC name 17β-Hydroxyandrost-4-en-3-one.

“Vitamin” means an organic molecule is essential to an organism in small quantities for proper metabolic function and which cannot be synthesized in the organism in sufficient quantities for survival, and which therefore must be obtained through the diet or other means of administration. Vitamins are usually classified as either water soluble vitamins or fat soluble vitamins. Fat soluble vitamins include vitamins A, D, E, and K. “Water soluble vitamins” typically refers to one or more of the following: vitamin C, vitamin B₁ (also known as

“thiamin”), vitamin B₂ (also known as “riboflavin”), vitamin B3 (also known as “niacin”), vitamin B₅ (also known as “pantothenic acid”), vitamin B₆, vitamin B₇ (also known as “biotin”), vitamin B9 (also known as “folate”) and vitamin B₁₂.

In one embodiment, the present disclosure provides, a chemical formulation comprising a vitamin, diarylheptanoid, piperidine, piperidine analog, flavonoid or steroid hormone combined with magnesium stearate or a salt with comparable properties to magnesium stearate.

In certain aspects, the formulation also contains a liposomal carrier complex.

In certain aspects, the formulation is an orally delivered drug.

In certain aspects, the formulation is a drug that is delivered nasally, by skin application, subcutaneously or as a suppository.

In certain aspects, the formulation comprises a solid dry powder.

In certain aspects, wherein the diarylheptanoid or flavonoid is curcumin, apigenin, luteolin and/or quercetin or a curcumin, apigenin, luteolin and/or quercetin analog.

In certain aspects, the steroid hormone is testosterone or a testosterone analog or an ester of testosterone, or a precursor of testosterone, or the molecule dehydroepiandrosterone, optionally further comprising piperine.

In certain aspects, the formulation contains magnesium stearate at a concentration of at least about 5%, 10%, 11%, 12%, 13%, 14%. 15%, 16%, 17%, 18%, 19%, 20%, 21%, 22%, 23%, 24%, or 25% w/w or 5% to 25% w/w. In some embodiments, the effective concentration of magnesium stearate (or a salt with comparable properties to magnesium stearate) in the formulation is within the range 5-25% w/w, or in a range bracketed by any two values listed above. In certain aspects, the present disclosure provides a chemical formulation comprising a vitamin combined with magnesium stearate or a salt with comparable properties to magnesium stearate. The vitamin can be a water-soluble vitamin or a fat-soluble vitamin.

In certain aspects, the formulation is used to treat an inflammatory disease.

In certain aspects, the formulation is used to treat lethargy, depression, mental stress, or anxiety, and/or Attention Deficit Hyperactivity Disorder (ADHD), or to enhance Attentional Control.

In certain aspects, the formulation is used to treat low libido.

In certain aspects, the formulation is used to treat obesity.

In another embodiment, the disclosure provides a method of treating inflammation comprising dosing a human patient with a drug comprising a chemical formulation containing flavonoid and magnesium stearate or a salt with comparable properties to magnesium stearate.

In certain aspects, the formulation also contains a liposomal carrier complex.

In certain aspects, the diarylheptanoid or flavonoid is curcumin, apigenin, luteolin and/or quercetin or a curcumin, apigenin, luteolin and/or quercetin analog.

In certain aspects, the formulation is delivered orally.

In certain aspects, the formulation is delivered nasally, by skin application, intravenously, subcutaneously or as a suppository.

In certain aspects, the formulation comprises a solid powder.

In certain aspects, the formulation also contains a liposomal carrier complex.

In certain aspects, the formulation contains magnesium stearate at a concentration of at least about 5%, 10%, 11%, 12%, 13%, 14%. 15%, 16%, 17%, 18%, 19%, 20%, 21%, 22%, 23%, 24%, or 25% w/w or 5% to 25% w/w.

In yet another embodiment, the disclosure provides a method for enhancing Attentional Control or for treating obesity, lethargy depression, mental stress or anxiety, ADHD, low libido, symptoms of peri-menopause and/or menopause, and/or inflammation, and/or providing hormonal support during fertility treatment, comprising dosing a human patient with a drug comprising a chemical formulation containing a steroid hormone and magnesium stearate.

In certain aspects, the formulation also contains a liposomal carrier complex.

In certain aspects, the steroid hormone is testosterone or a testosterone analog optionally further comprising piperine.

In certain aspects, the formulation is delivered orally.

In certain aspects, the formulation is delivered intravenously.

In certain aspects, the formulation comprises a solid powder.

In certain aspects, the formulation also contains a liposomal complex carrier.

In certain aspects, the formulation contains magnesium stearate or a salt with comparable properties to magnesium stearate, at a concentration of at least about: 5%, 10%, 11%, 12%, 13%, 14%, 15%, 16%, 17%, 18%, 19%, 20%, or 25% or 5% to 25% w/w. In some embodiments, the effective concentration of magnesium stearate (or a salt with comparable properties to magnesium stearate) in the formulation is within the range of 5-25% w/w.

In another embodiment, the disclosure provides a chemical formulation comprising a combination of (i) curcumin, apigenin, luteolin kaempferol, and/or quercetin, (ii) testosterone, an estrogen, progesterone, or DHEA, (iii) a liposomal complex carrier and (iv) magnesium stearate or a salt with comparable properties to magnesium stearate.

In certain aspects, the formulation is an orally delivered drug.

In certain aspects, the formulation is a drug that is delivered nasally, by skin application, subcutaneously or as a suppository.

In certain aspects, the formulation comprises a solid dry powder.

In certain aspects, the formulation contains magnesium stearate, or a salt with comparable properties to magnesium stearate, at a concentration of at least about: 5%, 10%, 11%, 12%, 13%, 14%, 15%, 16%, 17%, 18%, 19%, 20%, or 25% or 5% to 25% w/w. In some embodiments, the effective concentration of magnesium stearate (or a salt with comparable properties to magnesium stearate) in the formulation is within the range 5-25% w/w, or in a range bracketed by any two values listed above.

In yet another embodiment, the disclosure provides an oil-based drug formulation comprising both testosterone, or an ester of testosterone, or a testosterone analog, and curcumin, or a curcumin analog, or a flavonoid, optionally further comprising piperine, which is delivered by injection.

In certain aspects, the formulation is a drug that is used to treat low libido and/or obesity and/or ADHD, and/or lethargy and/or Irritable Bowel Syndrome (IBS), and/or symptoms of peri-menopause and/or menopause, and/or inflammation, or to provide hormonal support during fertility treatment, or to enhance Attentional Control, which treatment has a reduced risk of cardiovascular disease as compared to control treatments comprising the formulation without curcumin, apigenin, luteolin and/or quercetin.

In certain aspects, the formulation is a drug that is used to treat low libido and/or obesity and/or ADHD, and/or IBS, or another inflammatory disease.

In yet another embodiment, the disclosure provides a method for increasing the Area Under Curve (AUC) of a biologically active steroid hormone drug or a diarylheptanoid or a flavonoid molecule or a vitamin, the method comprising combining magnesium stearate, or salt with comparable properties to magnesium stearate, into a formulation containing the biologically active hormone or flavonoid and a liposomal carrier, and delivering the drug to a human or animal subject, whereby the AUC is increased as compared to a subject treated with a control formulation lacking the magnesium stearate or its comparable salt.

In certain aspects, the steroid hormone is testosterone, an estrogen, progesterone, or DHEA.

In certain aspects, the diarylheptanoid or flavonoid is curcumin, apigenin, luteolin and/or quercetin.

In certain aspects, the delivery method is oral, nasal, intravenous, subcutaneous, by skin application, or by suppository.

In certain aspects, the formulation is a solid powder.

In certain aspects, the formulation contains magnesium stearate or a salt with comparable properties at a concentration of at least about: 5%, 10%, 11%, 12%, 13%, 14%, 15%, 16%, 17%, 18%, 19%, 20%, or 25% w/w. In some embodiments, the effective concentration of magnesium stearate (or a salt with comparable properties to magnesium stearate) in the formulation is within the range 5-25% w/w, or in a range bracketed by any two values listed above.

In certain aspects, the present disclosure provides chemical formulations that comprise a diarylheptanoid, flavonoid, or a vitamin or steroid hormone, and a salt or salts that function in a manner comparable to magnesium stearate, that is, a salt or salts with comparable properties to magnesium stearate. The salt or salts with said comparable properties may be selected from the group consisting of magnesium stearate, magnesium laurate, magnesium palmitate, magnesium myristate, calcium stearate, calcium laurate, calcium palmitate, calcium myristate, sodium stearate, sodium laurate, sodium palmitate, sodium myristate, sodium stearyl fumarate, zinc stearate, zinc laurate, zinc palmitate, zinc myristate, or salts derived from any one of stearic acid, lauric acid, palmitic acid, and myristic acid.

Several of the foregoing salts are commonly used for pharmaceuticals including magnesium stearate, salts of stearic acid, and sodium stearyl fumarate.

Formulations of this disclosure may comprise: a diarylheptanoid, flavonoid or steroid hormone combined with magnesium stearate or a salt with comparable properties to magnesium stearate.

Concentration ranges of magnesium stearate or a salt with comparable properties to magnesium stearate can be at a concentration of at least 5%, 10%, 11%, 12%, 13%, 14%, 15%, 16%, 17%, 18%, 19%, 20%, 21%, 22%, 23%, 24%, or 25% w/w; or about 5%, 10%, 11%, 12%, 13%, 14%, 15%, 16%, 17%, 18%, 19%, 20%, 21%, 22%, 23%, 24%, or 25% w/w, or in a range of 5% to 10%, 10% to 20%, 20% to 30%, 30% to 40%, 40% to 50%, 50% to 60%, 60% to 70%, 70% to 80%, 80% to 90%, or 90% to 96%, or in a range bracketed by any two values listed above.

Testosterone can be at a concentration of at least 2 g, 3 g, 4 g, 5 g, 6 g, 7 g, 8 g, 9 g, 10 g, 11 g, 12 g, 13 g, 14 g, 15 g, 16 g, 17 g, 18 g, 19 g, 20 g, 21 g, 22 g, 23 g, 24 g, 25 g, 26 g, 27 g, 28 g, 29 g, 30 g, 31 g, 32 g, 33 g, 34 g, 35 g, 36 g, 37 g, 38 g, 39 g, 40 g, 41 g, 42 g, 43 g, 44 g, 45 g, 46 g, 47 g, 48 g, 49 g, 50 g, 51 g, 52 g, 53 g, 54 g, 55 g, 56 g, 57 g, 58 g, 59 g, 60 g, 61 g, 62 g, 63 g, 64 g, 65 g, 66 g, 67 g, 68 g, 69 g, 70 g, 71 g, 72 g, 73 g, 74 g, 75 g, 76 g, 77 g, 78 g, 79 g, or 80 g per 100 g of total formulation, or in a range of 2 g to 5 g, 5 g to 10 g, 10 g to 20 g, 20 g to 30 g, 30 g to 40 g, 40 g to 50 g, 50 g to 60 g, 60 g to 70 g, or 70 g to 80 g per 100 g of total formulation,, or in a range bracketed by any two values listed above.

Lecithin can be at a concentration of at least 2 g, 3 g, 4 g, 5 g, 6 g, 7 g, 8 g, 9 g, 10 g, 11 g, 12 g, 13 g, 14 g, 15 g, 16 g, 17 g, 18 g, 19 g, 20 g, 21 g, 22 g, 23 g, 24 g, 25 g, 26 g, 27 g, 28 g, 29 g, 30 g, 31 g, 32 g, 33 g, 34 g, 35 g, 36 g, 37 g, 38 g, 39 g, 40 g, 41 g, 42 g, 43 g, 44 g, 45 g, 46 g, 47 g, 48 g, 49 g, 50 g, 51 g, 52 g, 53 g, 54 g, 55 g, 56 g, 57 g, 58 g, 59 g, 60 g, 61 g, 62 g, 63 g, 64 g, 65 g, 66 g, 67 g, 68 g, 69 g, 70 g, 71 g, 72 g, 73 g, 74 g, 75 g, 76 g, 77 g, 78 g, 79 g, 80 g, 81 g, 82 g, 83 g, 84 g, 85 g, 86 g, 87 g, or 88 g per 100 g of total formulation, or in a range of 2 g to 5 g, 5 g to 10 g, 10 g to 20 g, 20 g to 30 g, 30 g to 40 g, 40 g to 50 g, 50 g to 60 g, 60 g to 70 g, 70 g to 80 g, or 80 g to 88 g per 100 g of which at least 30% consists of or comprises phospholipids, or in a range bracketed by any two values listed above.

In certain aspects, the formulations herein show improvements and differentiating features of formulations of curcumin disclosed herein versus existing formulations.

For example, a product containing curcumin is offered for sale on the following website: vs-corp.com/ip/; and references patent patents. google. com/patent/US9,192,644B2/en? oq=9192644.

The product specified herein differs from the art in the following ways:
the use case is different (reduction in low grade systemic inflammation in our claim vs treatment of Alzheimer's disease for the granted patent listed above)-this has been shown to be an effect of curcumin in vitro, and to some extent in humans, although hitherto, low bioavailability has hindered efficacy in humans;
the product described herein has different ingredients and is distinguished by the addition of a hydrophobic component;
the preparation method detailed herein is different (the method herein may rely on preparation of a solid dry powder that does not require excipients for production incapsule form).

In certain aspects, the present disclosure provides a solid formulation containing a phospholipid carrier and magnesium stearate.

A phospholipid carrier complex in a solid formulation may comprise greater than 10% by weight of magnesium stearate as a hydrophobic component to prevent or retard the absorption of water from the air or surrounding environment during storage.

In certain aspects, the phospholipid carrier complex comprises 10%-20% by weight of magnesium stearate.

In certain aspects, the phospholipid carrier complex comprises lecithin, phosphatidylcholine, and/or bile salts.

In another embodiment, the disclosure provides a method of increasing absorption and bioavailability of a bioactive chemical to increase the AUC of the bioactive chemical in the bloodstream.

In certain aspects, the method comprises the steps of combining 10%-60% by weight of magnesium stearate in a formulation with lecithin, phosphatidylcholine, and/or bile salts in a formulation combination with the bioactive molecule and delivering said formulation to a human or animal subject by oral delivery or by injection.

In certain aspects, the biochemical active in the formulation is a curcuminoid that reduces chronic low-grade inflammation in a human.

In certain aspects, the formulation increases the absorption of curcumin and the flavonoid quercetin into intestinal epithelial cells to reduce the generic symptoms of inflammatory bowel syndrome (IBS) or disorders.

In certain aspects, the formulation increases the AUC of flavonoids in the bloodstream.

In certain aspects, flavonoids are apigenin or/or luteolin.

In certain aspects, the formulation reduces mental stress or anxiety, improves mental acuity and/or reduces symptoms of ADHD in a human or an animal.

In certain aspects, the formulation increases the AUC of testosterone.

In certain aspects, the testosterone has a purity of at about 90%, 91%, 92%, 93%, 94%, 95%, 96%, 97%, 98%, 99% or 100%, or in a range bracketed by any two values listed above.

In another embodiment, the disclosure provides a use of an oil-based carrier to lubricate a glycerol-based injection. Since injectable oils are typically food oils which are essentially immiscible with glycerol and also much less viscous, the oil component may serve to lubricate the injection apparatus (e.g. syringe and needle) allowing for easier injection than if a viscous glycerol-based solution is injected alone. Furthermore, the oil may create a pocket that contains the glycerol-based solution and, due to the immiscibility of the oil, prevent the glycerol solution from excessive direct contact with tissues at the injection site, preventing cytotoxicity and allowing for slow, steady dissolution of the glycerol solution into the lymph and blood stream.

In certain aspects, the disclosure provides applications to other steroid molecules including steroid sex hormones.

In certain aspects, the formulations, products, assessments, methods, and uses set forth herein can further include other types of steroid molecule such as estrogens, progesterone, and potential analogs and/or precursors of steroid hormones including DHEA/dehydroepiandrosterone.

In certain aspects, the examples, formulations, products, assessments, methods, and uses set forth herein can be further extended to include piperidine, a homolog of piperidine or another CYP3A4 inhibitor.

In certain aspects, the steroid formulation comprises piperine or a derivative thereof combined with magnesium stearate or a salt with comparable properties to magnesium stearate and a liposomal carrier complex, along with testosterone (or another steroid molecule). Piperine (an extract from black pepper) or other CYP3A4 antagonist in combination with testosterone or other steroid hormones in the same formulation. CYP3A4 is the primary enzyme involved in the breakdown of testosterone in the body by the liver. Reducing the function of this enzyme, at the same time as increasing bioavailability of testosterone by use of this current formulation increases testosterone circulation levels more than the same formulation without piperine.

In still yet another embodiment, the present disclosure provides a different active ingredient in the formulation, i.e., other than steroid molecules. For example, a vitamin can be used in the present formulation vehicle. Vitamins can be divided into water-soluble vitamins and fat-soluble vitamins.

In certain aspects, the formulation comprises a water-soluble vitamin combined with magnesium stearate or a salt with comparable properties to magnesium stearate and a liposomal carrier complex.

Water soluble vitamins are readily bioavailable. Because of their solubility, however, they are easily lost in urine and fecal matter, and therefore a dose taken as a vitamin supplement may have far less effect than expected. Vitamin formulations of the disclosure will delay the release of water-soluble vitamins into a free form in the bloodstream, thereby increasing their duration of action and their half-life. Water-soluble vitamins include the B vitamins, folate, thiamine, riboflavin, niacin, pantothenic acid, biotin, vitamin B₆, and vitamin B₁₂; and vitamin C. Deficiency of any of these water-soluble vitamins results in a clinical syndrome that may result in severe morbidity and mortality.

Fat soluble vitamins do not have the same issue. Humans with pancreatic deficiency may experience deficiencies in these fat-soluble vitamins due to their restricted ability to absorb fats, and the fat-soluble vitamins that are associated with fat. Fat-soluble vitamins contained within this current formulation are absorbed directly without the need for endogenous micelles or liposomes that require pancreatic function for generation. Fat-soluble vitamins include Vitamin A, Vitamin D, Vitamin E and Vitamin K.

In one embodiment of the invention, the practitioner selects a molecule of interest from the group consisting of diarylheptanoid, flavonoid, vitamin and steroid hormone to produce a beneficial formulation.

In some embodiments, the selected molecule of interest is soluble in organic solvents (e.g., ethanol). The manufacture step typically begins with blending three components: a liposomal carrier complex, such as lecithin, phosphatidylcholine, and/or bile salts magnesium stearate (or a salt with comparable properties to magnesium stearate) and with the selected molecule of interest in dry form. Following the dry blend, an organic solvent is then introduced to the mixture to a uniform homogeneous slurry. Typically ethanol is used as the organic solvent, but other Nonlimiting examples of organic solvents that may be used include Ethanol, Methanol, Isopropanol (IPA), 3-Methyl-1-butanol, Acetone, Methylethyl ketone (MEK/ 2-Butanone), Methylbutyl ketone, Ethyl acetate, Isopropyl acetate, Isobutyl acetate, Dichloromethane (Methylene chloride), Chloroform, Diethyl ether, 1,4-Dioxane, Tetrahydrofuran (THF), Acetonitrile, Dimethyl sulfoxide (DMSO), Dimethylformamide (DMF), Hexane, Heptane, Toluene, and Cyclohexane.

This solvent-assisted blending enables efficient mixing and the components to reside in close molecular proximity. Efficient mixing is typically indicated by the distinct physical phase change of magnesium stearate. During the process, the magnesium stearate (or salt with comparable properties) changes its physical appearance from an initial state of discrete “waxy flakes” into a state where it is physically indistinguishable from the other components of the mixture and cannot be readily separated. The solvent is then removed by evaporation, resulting in a solid, uniform mass. The mass is then pulverized into a powder suitable for encapsulation.

In some embodiments, the selected molecule of interest is water-soluble (e.g. a water soluble vitamin), the molecule is dissolved in an aqueous solution. The solution is added to a mixture of magnesium stearate (or salt with similar properties) and the liposomal carrier, to which an organic solvent (e.g., ethanol) has already been added to form a mixture, optionally with stirring to enhance efficient mixing. A homogenous slurry is again formed and the water and organic solvent are then removed via evaporation to leave a solid mass, which is further processed and delivered as described above.

The resulting encapsulated formulation is designed for oral delivery, allowing the formulation to bypass the stomach of the subject and to enter the intestines. Upon reaching the aqueous environment of the intestines, the magnesium stearate (or salt with comparable properties to magnesium stearate) component of the formulation, being hydrophobic, separates from the liposomal carrier and the molecule of interest. The active molecule being in close proximity to the liposomal carrier, incorporates into liposomes, which form spontaneously, and which are then absorbed through the epithelium of the gut, bypassing digestion and enabling the active molecule to be bioavailable.

### EXAMPLES

Example 1: Inclusion of magnesium stearate in a formulation containing testosterone and lecithin.

Test Formulations were prepared comprising magnesium stearate comprising about 10 g, or about 11 g or about 12 g, or about 13 g, or about 14 g or about 15 g or about, or about 16 g or about, or about 17 g or about, or about 18 g, or about 19 g, or about 20 g per 100 g of total formulation, and which also contained testosterone (about 2 g and up to 80 g per 100 g of total formulation) and lecithin (about 2 g and up to 88 g per 100 g of total formulation of which at least 30 % consists of phospholipids). The formulations were visibly finer and less prone to clumping and agglomeration compared to a control formulation lacking magnesium stearate, after 7 days of leaving it exposed to air. It was observed that the addition of magnesium stearate dramatically changed the physical characteristics of the formulations; without the magnesium stearate, the control formulations agglomerated into sticky mass. The formulations with the magnesium stearate present remained in the form of a very fine powder.

Example 2: Inclusion of magnesium stearate in a formulation containing curcumin and lecithin.

Test formulations were prepared comprising magnesium stearate comprising about 10 g, or about 11 g or about 12 g, or about 13 g, or about 14 g or about 15 g or about, or about 16 g or about, or about 17 g or about, or about 18 g, or about 19 g, or about 20 g per 100 g of total formulation, and which also contained curcumin (about 2 g and up to 80 g per 100 g of total formulation) and lecithin (at least 2 g and up to 88 g per 100 g of total formulation of which at least 30% consists of phospholipids). The formulations were visibly finer and less prone to clumping and agglomeration compared to a control formulation lacking magnesium stearate, after 7 days of leaving it exposed to air. It was observed that the addition of magnesium stearate dramatically changed the physical characteristics of the formulations; without the magnesium stearate, the control formulations agglomerated into sticky mass. The formulation with the magnesium stearate present remained in the form of a very fine powder.

Example 3: Inclusion of magnesium stearate in a formulation containing testosterone and phosphatidylcholine.

Test formulations were prepared comprising magnesium stearate comprising about 10 g, or about 11 g or about 12 g, or about 13 g, or about 14 g or about 15 g or about, or about 16 g or about, or about 17 g or about, or about 18 g, or about 19 g, or about 20 g per 100 g of total formulation, which also contained testosterone (at least 2 g and up to 80 g per 100 g of total formulation) and phosphatidylcholine (at least 2 g and up to 67 g per 100 g of total formulation). The formulations were visibly finer and less prone to clumping and agglomeration compared to a control formulation lacking magnesium stearate, after 5 days of leaving it exposed to air. It was observed that the addition of magnesium stearate dramatically changed the physical characteristics of the formulations; without the magnesium stearate, the control formulations agglomerated into sticky mass. The formulations with the magnesium stearate present, remained in the form of a very fine powder.

Example 4: Inclusion of magnesium stearate in a formulation containing curcumin and phosphatidylcholine.

Test formulations were prepared comprising magnesium stearate comprising about 10 g, or about 11 g or about 12 g, or about 13 g, or about 14 g or about 15 g or about, or about 16 g or about, or about 17 g or about, or about 18 g, or about 19 g, or about 20 g per 100 g of total formulation, which also contained curcumin (about 2 g and up to 80 g per 100 g of total formulation) and phosphatidylcholine (about 2 g and up to 67 g per 100 g of total formulation). The formulations were visibly finer and less prone to clumping and agglomeration compared to a control formulation lacking magnesium stearate, after 5 days of leaving it exposed to air. It was observed that the addition of magnesium stearate dramatically changed the physical characteristics of the formulations; without the magnesium stearate, the control formulations agglomerated into sticky mass. The formulations with the magnesium stearate present, remained in the form of a very fine powder.

Example 5: A simple dispersibility test to model the dispersion of a drug within the body of a subject.

The following method can be used. Stir a given amount of powdered formulation in distilled water at 37 degrees Centigrade. Allow the suspension to settle for a period of 5 minutes at and visibly observe the amount of suspended material versus sediment. Alternatively, the amount of suspended material may be quantified by an IR refraction-based method.

The formulations described in examples 1-4 were suspended in water according to the above method. In each instance, approximately 10 g of the total formulation was added to 100 ml of distilled water a visibly greater quantity of the formulation remained in the aqueous fraction after a five-minute periodin the formulations containing magnesium stearate, as compared to the control formulations lacking magnesium stearate. Based on these results, it was concluded that the addition of magnesium stearate likely enhances the bioavailability of the active ingredients within a human or animal subject.

Example 6: A formulation comprising both Quercetin and Curcumin for the treatment of IBS.

A formulation is provided to subjects which includes both Quercetin (at least 2 g and up to 80 g per 100 g of total formulation) and Curcumin (at least 2 g and up to 80 g per 100 g of total formulation) in combination with either bile salts, (at least 2 g and up to 67 g per 100 g of total formulation), phosphatidylcholine (at least 2 g and up to 67 g per 100 g of total formulation) and/or lecithin (at least 2 g and up to 86 g per 100 g of total formulation of which at least 30% consists of phospholipids) in combination with magnesium stearate at rate of at least 10 g, or at least 11 g or at least 12 g, or at least 13 g, or at least 14 g or at least 15 g or at least, or at least 16 g or at least, or at least 17 g or at least, or at least 18 g, or at least 19 g, or at least 20 g per 100 g of total formulation. The formulation is provided to one or more subjects and control formulations lacking one or more components are provided to one or more subjects, as part of an observational study. The formulation is concluded to have a benefit in reducing IBS symptoms, as assessed by means of questionnaire with IBS symptoms diagnosed according to the Rome Criteria questionnaire.

Example 7: A formulation comprising both Luteolin and Apigenin for the treatment of ADHD.

A formulation is provided to subjects which includes both Apigenin (at least 2 g and up to 80 g per 100 g of total formulation) and Luteolin (at least 2 g and up to 80 g per 100 g of total formulation) in combination with either bile salts, (at least 2 g and up to 67 g per 100 g of total formulation), phosphatidylcholine (at least 2 g and up to 67 g per 100 g of total formulation) and/or lecithin (at least 2 g and up to 86 g per 100 g of total formulation of which at least 30% consists of phospholipids) in combination with magnesium stearate at rate of at least 10 g, or at least 11 g or at least 12 g, or at least 13 g, or at least 14 g or at least 15 g or at least, or at least 16 g or at least, or at least 17 g or at least, or at least 18 g, or at least 19 g, or at least 20 g per 100 g of total formulation. The formulation is provided to one or more subjects, and control formulations lacking one or more components are provided to one or more subjects, as part of an observational study and it is concluded to have a benefit in reducing symptoms of ADHD or improving concentration as assessed by means of questionnaire. The efficacy of the combination formulation is also assessed by subjecting subjects to objective task completion tests.

Example 8: Assessment of the effectiveness of curcumin in reducing inflammation.

Blood testing is performed to evaluate common biomarkers of inflammation at fixed periods before and after taking a course of curcumin delivered in the formulations specified in examples 2 and/or 4, above, as compared to unformulated and control equivalents.

Example 9: Assessment of the availability of Testosterone administered according to examples 1 or 3 above by oral or sublingual route.

AUC blood testing is performed by means of measuring total testosterone via a standard blood test that is readily available clinically.

Example 10: Addition of a curcuminoid and/or a flavonoid to a formulation offset the potential adverse effects of testosterone.

Formulations comprising a combination of testosterone and/or a curcuminoid and/or a flavonoid are hereby disclosed. There has been extensive discussion in medical literature regarding a link between testosterone therapy and increased cardiovascular disease risk. This led the FDA to release a warning statement about the potential cardiovascular risks of testosterone replacement therapy (Gagliano-Jucá and Basaria, 2019, Nat Rev Cardiol, 16(9), 555-574). Curcumin and certain flavonoids, including but not limited to resveratrol and certain anthocyanins, are believed to protect against heart disease and other cardiovascular ailments. A formulation, as provided herein, which includes a curcuminoid and/or a flavonoid in combination with testosterone (or a testosterone analog) therefore provides a means to mitigate this potential risk of poor cardiovascular health. The combination formulation can be provided either as an injectable oil-based preparation or as an orally delivered powder.

The efficacy of the above formulation can be measured via blood testing to evaluate the effect on common biomarkers of inflammation at fixed periods before and after taking a course of this formulation of curcuminoid combined with testosterone vs the formulation of testosterone alone.

Some common biomarkers, which can be used to measure inflammation include: blood counts (e.g., white blood cell count, red blood cell count, hemoglobin count), C-reactive protein (CRP), Erythrocyte sedimentation rate (ESR), Procalcitonin and Calprotectin.

Example 11: Products based on formulations and specific use cases.

Five specific use cases exist of the methods disclosed herein for increasing absorption and bioavailability of bioactive chemicals. These are listed as (a)-(d) below:
(a) to increase the AUC of curcumin in the bloodstream. The specific use of this product is to reduce systemic, chronic low-grade inflammation in humans and/or animals.
(b) to increase the absorption of curcumin and the flavonoid quercetin into intestinal epithelial cells to reduce the generic symptoms of IBS and similar conditions.
(c) to increase the AUC of the flavonoids apigenin and luteolin in the bloodstream. The specific example uses for this are reduction of mental stress or anxiety, to increase concentration and mental acuity, and/or to reduce symptoms of ADHD.
(d) to increase the AUC of pure testosterone. Testosterone is commonly used as male hormone replacement therapy (as well as to suppress female secondary sexual characteristics in transsexual men). However, testosterone is not currently used orally other than as esters, and is in fact most commonly administered by injection. The formulations presented herein may enable physiologically significant levels of testosterone in the bloodstream to be achieved by means of oral administration of the formulations.
(e) to improve the retention of beneficial vitamins by a human or animal body. Water soluble vitamins are lost from the body relatively rapidly if administered as a bolus; the formulations detailed herein will improve retention times in the body and increase the benefits from administration of such molecules. A specific application would be individuals with pancreatic deficiency; delivery of vitamins in the formulations detailed herein may enhance the absorption of such molecules by these patients.

Example 12: Preparation of a High-Bioavailability Testosterone Formulation

In a representative embodiment, 100 mg of testosterone (as the molecule of interest) is combined in dry form with 200 mg of lecithin and 50 mg of magnesium stearate. The dry components are blended to ensure initial distribution. Following the dry blend, approximately 5 mL of 95% ethanol is introduced to the mixture. The mixture is stirred vigorously until a uniform, homogeneous slurry is formed. During this stage, the magnesium stearate transitions from discrete waxy flakes into a state where it is physically indistinguishable from the lecithin and testosterone at a macro-scale. The ethanol is then removed via rotary evaporation at 40° C., leaving a solid uniform mass. This mass is pulverized into a fine powder and encapsulated in a HPMC capsule. Upon oral administration, the molecular proximity established during the slurry phase allows for spontaneous liposome formation in the intestinal tract, significantly increasing the testosterone Area Under Curve (AUC) compared to standard micronized testosterone.

Example 13: Preparation of a High-Bioavailability Water-Soluble Vitamin Formulation

In another embodiment, 50 mg of a water-soluble vitamin (e.g., Vitamin C or B12) is dissolved in 2 mL of deionized water to form an aqueous solution. Separately, 200 mg of lecithin and 50 mg of magnesium stearate are mixed with 3 mL of ethanol. The aqueous vitamin solution is then added to the ethanol-carrier mixture with continuous stirring to form a homogeneous slurry. The combined solvents (water and ethanol) are removed under vacuum, resulting in a solid uniform mass where the vitamin is held in close molecular proximity to the lecithin carrier. The mass is pulverized and encapsulated. This method enables the water-soluble vitamin to bypass primary gastric digestion and achieve enhanced absorption through the gut epithelium via spontaneously formed liposomes.

Example 14 Tests of formulations on a human subject

The following combinations were mixed in dry form (i.e., solid form) and then mixed with ethanol until a homogeneous slurry was obtained. The ethanol was evaporated, and the solid mixture was pulverized and encapsulated:
i. 2 g Magnesium stearate, 8 g Lecithin, 90 g testosterone
ii. 2 g Magnesium stearate, 8 g Phosphatidyl choline, 90 g testosterone
iii. 2 g Magnesium stearate, 8 g Lecithin, 90 g curcumin
iv. 2 g Magnesium stearate, 8 g Phosphatidyl choline, 90 g curcumin
v. 5 g Magnesium stearate, 15 g Lecithin, 80 g testosterone
vi. 5 g Magnesium stearate, 15 g Phosphatidyl choline, 80 g testosterone
vii. 5 g Magnesium stearate, 15 g Lecithin, 80 g curcumin
viii. 5 g Magnesium stearate, 15 g Phosphatidyl choline, 80 g curcumin
ix. 10 g Magnesium stearate, 20 g Lecithin, 70 g testosterone
x. 10 g Magnesium stearate, 20 g Phosphatidyl choline, 70 g testosterone
xi. 10 g Magnesium stearate, 20 g Lecithin, 70 g curcumin
xii. 10 g Magnesium stearate, 20 g Phosphatidyl choline, 70 g curcumin
xiii. 33 g Magnesium stearate, 33 g Lecithin, 33 g testosterone
xiv. 33 g Magnesium stearate, 33 g Phosphatidyl choline, 33 g testosterone
xv. 33 g Magnesium stearate, 33 g Lecithin, 33 g curcumin
xvi. 33 g Magnesium stearate, 33 g Phosphatidyl choline, 33 g curcumin
xvii. 40 g Magnesium stearate, 40 g Lecithin, 20 g testosterone
xviii. 40 g Magnesium stearate, 40 g Phosphatidyl choline, 20 g testosterone
xix. 40 g Magnesium stearate, 40 g Lecithin, 20 g curcumin
xx. 40 g Magnesium stearate, 40 g Phosphatidyl choline, 20 g curcumin
xxi. 45 g Magnesium stearate, 45 g Lecithin, 10 g testosterone
xxii. 45 g Magnesium stearate, 45 g Phosphatidyl choline, 10 g testosterone
xxiii. 33 g Magnesium stearate, 33 g Lecithin, 20 g lutein, 20 g apigenin
xxiv. 33 g Magnesium stearate, 33 g Phosphatidyl choline, 20 g lutein, 20 g apigenin

Three capsules of each of the above formulations were taken orally by a 50 year old male human subject at approximately 8 am on different days, 2 days apart. Each formulation was tested on two different days. Each capsule contained 500 mg-800 mg of dry powdered formation. In each case, the male subject was questioned approximately 8 hours after consumption of the capsules and asked whether he had suffered any ill effects such as gastric pain or diarrhea and asked whether he had felt any change in mood or vigor. The subject reported no ill effects with any of the combinations tested. He noted that following consumption of the capsules containing testosterone, and the lutein apigenin combination, in all but two tests, he felt in a more buoyant mood and felt generally more energetic than on the days when no formulation was consumed. The exceptions were the formulations containing only 10% (w/w) testosterone in the total mixture, for which the subject reported feeling no perceptible difference in mood or energy levels. The subject reported no ill effects from consumption of the curcumin containing capsules, but did not note any effect on mood or energy levels in these cases.

### EXEMPLARY EMBODIMENTS

This Disclosure Includes the Following Exemplary Embodiments:
1. A chemical formulation comprising a diarylheptanoid, flavonoid, vitamin or steroid hormone combined with magnesium stearate or a salt with comparable properties to magnesium stearate. Optionally, the chemical formulation is in solid form.
2. The formulation of embodiment 1, wherein the formulation also contains a liposomal carrier complex.
3. The formulation according to embodiments 1 or 2, wherein the formulation is an orally delivered drug.
4. The formulation according to embodiments 1 or 2, wherein the formulation is a drug that is delivered nasally, by skin application, subcutaneously or as a suppository.
5. The formulation according to embodiments 1 or 2, wherein the formulation comprises a solid dry powder.
6. The formulation according to embodiments 1, 2, 3, 4, or 5, wherein the diarylheptanoid or flavonoid is curcumin, apigenin, luteolin and/or quercetin or a curcumin, apigenin, luteolin and/or quercetin analog.
7. The formulation according to embodiments 1, 2, 3, 4, or 5, wherein the steroid hormone is testosterone or a testosterone analog or an ester of testosterone, or a precursor of testosterone, or the molecule dehydroepiandrosterone optionally further comprising piperidine, a piperidine analog, or another CYP3A4 inhibitor.
8. The formulation according to embodiments 1, 2, 3, 4, 5, 6, or 7, wherein the formulation contains magnesium stearate at a concentration in a range of 5% to 25% (w/w), for example, about 5%, 10%, 11%, 12%, 13%, 14%. 15%, 16%, 17%, 18%, 19%, 20%, 21%, 22%, 23%, 24%, or 25% w/w.
9. The formulation according to embodiments 1, 2, 3, 4, 5, 6, 7, or 8, wherein the formulation is used to treat an inflammatory disease.
10. The formulation according to embodiments 1, 2, 3, 4, 5, 6, 7, or 8, wherein the formulation is used to treat lethargy, depression, mental stress, or anxiety, and/or Attention Deficit Hyperactivity Disorder (ADHD), or to enhance Attentional Control.
11. The formulation according to embodiments 1, 2, 3, 4, 5, 6, 7, or 8, wherein the formulation is used to treat low libido.
12. The formulation according to embodiments 1, 2, 3, 4, 5, 6, 7, or 8, wherein the formulation is used to treat obesity.
13. A method of treating inflammation comprising dosing a human patient with a drug comprising a chemical formulation containing flavonoid and magnesium stearate or a salt with comparable properties to magnesium stearate.
14. The method of embodiment 13, wherein the formulation also contains a liposomal carrier complex.
15. The method according to embodiments 13 or 14, wherein the diarylheptanoid or flavonoid is curcumin, apigenin, luteolin and/or quercetin or a curcumin, apigenin, luteolin and/or quercetin analog.
16. The method according to embodiments 13, 14, or 15, wherein the formulation is delivered orally.
17. The method according to embodiments 13, 14, or 15, wherein the formulation is delivered nasally, by skin application, intravenously, subcutaneously or as a suppository.
18. The method according to embodiments 13, 14, or 15, wherein the formulation comprises a solid powder.
19. The method according to embodiments 13, 14, or 15, wherein the formulation also contains a liposomal carrier complex.
20. The method according to embodiments 13, 14, 15, 16, 17, 18, or 19, wherein the formulation contains magnesium stearate at a concentration in a range of 5% to 25% (w/w), for example, about 5%, 10%, 11%, 12%, 13%, 14%. 15%, 16%, 17%, 18%, 19%, 20%, 21%, 22%, 23%, 24%, or 25% w/w.
21. A method for enhancing Attentional Control or for treating obesity, lethargy depression, mental stress or anxiety, ADHD, low libido, symptoms of peri-menopause and/or menopause, and/or inflammation, and/or providing hormonal support during fertility treatment, comprising dosing a human patient with a drug comprising a chemical formulation containing a steroid hormone and magnesium stearate.
22. The method of embodiment 21, wherein the formulation also contains a liposomal carrier complex.
23. The method according to embodiments 21 or 22, wherein the steroid hormone is testosterone or a testosterone analog optionally further comprising piperidine, a piperidine analog, or another CYP3A4 inhibitor.
24. The method according to embodiments 21, 22, or 23, wherein the formulation is delivered orally.
25. The method according to embodiments 21, 22, or 23, wherein the formulation is delivered intravenously.
26. The method according to embodiments 21, 22, or 23, wherein the formulation comprises a solid powder.
27. The method according to embodiments 21, 22, or 23, wherein the formulation also contains a liposomal complex carrier.
28. The method according to embodiments 21, 22, 23, 24, 25, 26, or 27, wherein the formulation contains magnesium stearate or a salt with comparable properties to magnesium stearate, at a concentration in a range of 5% to 25% (w/w), for example about: 5%, 10%, 11%, 12%, 13%, 14%, 15%, 16%, 17%, 18%, 19%, 20%, or 25% (w/w).
29. A chemical formulation comprising a combination of (i) curcumin, apigenin, luteolin kaempferol, and/or quercetin, (ii) testosterone, an estrogen, progesterone, or DHEA, (iii) a liposomal complex carrier and (iv) magnesium stearate or a salt with comparable properties to magnesium stearate.
30. The formulation of embodiment 29, wherein the formulation is an orally delivered drug.
31. The formulation of embodiment 29, wherein the formulation is a drug that is delivered nasally, by skin application, subcutaneously or as a suppository.
32. The formulation of embodiment 29, wherein the formulation comprises a solid dry powder.
33. The chemical formulation according to embodiments 30, 31 or 32, wherein the formulation contains magnesium stearate, or a salt with comparable properties to magnesium stearate, at a concentration in a range of 5% to 25% (w/w), for example about: 5%, 10%, 11%, 12%, 13%, 14%, 15%, 16%, 17%, 18%, 19%, 20%, or 25% w/w.
34. An oil-based drug formulation comprising both testosterone, or an ester of testosterone, or a testosterone analog, and curcumin, or a curcumin analog, or a flavonoid, optionally further comprising piperine, which is delivered by injection.
35. The formulation according to embodiments 29, 30, 31, 32, 33, or 34, wherein the formulation is a drug that is used to treat low libido and/or obesity and/or ADHD, and/or lethargy and/or Irritable Bowel Syndrome (IBS), and/or symptoms of peri-menopause and/or menopause, and/or inflammation, or to provide hormonal support during fertility treatment, or to enhance Attentional Control, which treatment has a reduced risk of cardiovascular disease as compared to control treatments comprising the formulation without curcumin, apigenin, luteolin and/or quercetin.
36. The formulation according to embodiments 29, 30, 31, 32, 33, or 34, wherein the formulation is a drug that is used to treat low libido and/or obesity and/or ADHD, and/or IBS, or another inflammatory disease.
37. A method for increasing the Area Under Curve (AUC) of a biologically active steroid hormone drug or a diarylheptanoid or a flavonoid molecule, or a vitamin, the method comprising combining magnesium stearate, or salt with comparable properties to magnesium stearate, into a formulation containing the biologically active hormone or flavonoid and a liposomal carrier, and delivering the drug to a human or animal subject, whereby the AUC is increased as compared to a subject treated with a control formulation lacking the magnesium stearate or its comparable salt.
38. The method of embodiment 37, wherein the steroid hormone is testosterone, an estrogen, progesterone, or DHEA, optionally further comprising piperidine, a piperidine analog, or another CYP3A4 inhibitor.
39. The method of embodiment 37, wherein the diarylheptanoid or flavonoid is curcumin, apigenin, luteolin and/or quercetin.
40. The method according to embodiments 37, 38 or 39, wherein the delivery method is oral, nasal, intravenous, subcutaneous, by skin application, or by suppository.
41 The method according to embodiments 37, 38 or 39, wherein the formulation is a solid powder.
42. The method according to embodiments 37, 38 or 39, wherein the formulation contains magnesium stearate or a salt with comparable properties at a concentration in a range of 5% to 25% (w/w), for example, about: 5%, 10%, 11%, 12%, 13%, 14%, 15%, 16%, 17%, 18%, 19%, 20%, or 25% w/w.
43. A method for producing a chemical formulation comprising the steps of:
   (a) blending a dry mixture comprising a molecule of interest that is soluble in an organic solvent, a liposomal carrier complex, and magnesium stearate or a salt with comparable properties to magnesium stearate;
   (b) adding the organic solvent to the dry mixture and mixing to produce a uniform homogeneous slurry;
   (c) removing the organic solvent by evaporation to leave a solid uniform mass; and
   (d) pulverizing the solid uniform mass into a powder.
44. The method of embodiment 43, wherein the organic solvent is ethanol.
45. The method of embodiment 43, wherein the mixing in step (b) is performed until the magnesium stearate or salt with comparable properties changes physical appearance from waxy flakes to being indistinguishable as a separate component of the mixture.
46. A method for producing a chemical formulation containing a water-soluble molecule of interest, comprising the steps of:
   (a) dissolving the water-soluble molecule of interest in water to form an aqueous solution;
   (b) providing a mixture comprising a liposomal carrier complex and magnesium stearate or a salt with comparable properties to magnesium stearate;
   (c) adding an organic solvent and the aqueous solution of step (a) to the mixture of step (b) to produce a uniform homogeneous slurry;
   (d) removing the water and organic solvent by evaporation to leave a solid uniform mass; and
   (e) pulverizing the solid uniform mass into a powder.
47. The method according to embodiments 43 or 46, further comprising the step of encapsulating the powder into a capsule.
48. The method according to embodiments 43 or 46, wherein the liposomal carrier complex is lecithin.
49. The method according to embodiments 43 or 46, wherein the molecule of interest is selected from the group consisting of: a diarylheptanoid, a flavonoid, a vitamin, and a steroid hormone.

What is claimed is:

1. A chemical formulation comprising (i) a molecule of interest selected from the group consisting of a diarylheptanoid, flavonoid, vitamin or steroid hormone; (ii) a liposomal carrier complex; and (iii) magnesium stearate or a salt with comparable properties to magnesium stearate.

2. The formulation according to claim 1, wherein the formulation is a solid dry powder or an orally delivered drug.

3. The formulation of claim 1, wherein the diarylheptanoid or flavonoid is selected from the group consisting of: curcumin, apigenin, luteolin, quercetin, analogs thereof.4.

4. The formulation of claim 1, wherein the steroid hormone is selected from the group consisting of testosterone, a testosterone analog, an ester of testosterone, a precursor of testosterone, and dehydroepiandrosterone.

5. The formulation of claim 4, further comprising piperidine, a piperidine analog, or another CYP3A4 inhibitor.

6. The formulation of claim 1, wherein the formulation contains magnesium stearate at a concentration of about 5%, 10%, 11%, 12%, 13%, 14%. 15%, 16%, 17%, 18%, 19%, 20%, 21%, 22%, 23%, 24%, or 25% w/w.

7. The formulation of claim 1, wherein the formulation is used to treat an inflammatory disease, Irritable Bowel Syndrome (IBS), lethargy, depression, mental stress, or anxiety, and/or Attention Deficit Hyperactivity Disorder (ADHD), or to enhance Attentional Control, low libido, obesity, symptoms of peri-menopause, or symptoms of menopause.

8. A method of treating a subject in need thereof comprising administering to the subject a drug comprising a chemical formulation comprising a molecule of interest selected from the group consisting of flavonoid, a diarylheptanoid, and a steroid hormone, combined with a liposomal carrier complex, and magnesium stearate or a salt with comparable properties to magnesium stearate.

9. The method of claim 8, wherein the condition treated is selected from the group consisting of: inflammation, obesity, lethargy, depression, mental stress, anxiety, ADHD, low libido, symptoms of peri-menopause, and symptoms of menopause.

10. The method of claim 8, wherein the flavonoid is apigenin, luteolin, quercetin or an analog thereof.

11. The method of claim 8, wherein the steroid hormone is testosterone or a testosterone analog.

12. The method of claim 8, wherein the formulation is delivered orally, nasally, by skin application, intravenously, subcutaneously, or as a suppository.

13. The method of claim 8, wherein the formulation comprises a solid powder.

14. The method of claim 8, wherein the formulation contains magnesium stearate at a concentration of about 5%, 10%, 11%, 12%, 13%, 14%. 15%, 16%, 17%, 18%, 19%, 20%, 21%, 22%, 23%, 24%, or 25% w/w.

15. The method of claim 8, wherein the treatment results in an increased Area Under Curve (AUC) of the active molecule as compared to a subject treated with a control formulation lacking the magnesium stearate or its comparable salt.

16. A method for producing a chemical formulation comprising the steps of:
(a) blending a dry mixture comprising a molecule of interest that is soluble in an organic solvent, a liposomal carrier complex, and magnesium stearate or a salt with comparable properties to magnesium stearate;
(b) adding the organic solvent to the dry mixture and mixing to produce a uniform homogeneous slurry;
(c) removing the organic solvent by evaporation to leave a solid uniform mass; and
(d) pulverizing the solid uniform mass into a powder.

17. The method of claim 16, wherein the organic solvent is ethanol.

18. The method of claim 16, wherein the mixing in step (b) is performed until the magnesium stearate or salt with comparable properties changes physical appearance from waxy flakes to being indistinguishable as a separate component of the mixture.

19. The method of claim 16, further comprising the step of encapsulating the powder into a capsule.

20. The method of claim 16, wherein the liposomal carrier complex is lecithin.

21. The method of claim 16, wherein the molecule of interest is selected from the group consisting of: a diarylheptanoid, a flavonoid, a vitamin, and a steroid hormone.

22. A method for producing a chemical formulation containing a water-soluble molecule of interest, comprising the steps of:
(a) dissolving the water-soluble molecule of interest in water to form an aqueous solution;
(b) providing a mixture comprising a liposomal carrier complex and magnesium stearate or a salt with comparable properties to magnesium stearate;
(c) adding an organic solvent and the aqueous solution of step (a) to the mixture of step (b) to produce a uniform homogeneous slurry;
(d) removing the water and organic solvent by evaporation to leave a solid uniform mass; and
(e) pulverizing the solid uniform mass into a powder.

23. The method of claim 22, wherein the molecule of interest is selected from the group consisting of: a diarylheptanoid, a flavonoid, a vitamin, and a steroid hormone.

24. The method of claim 22, wherein the organic solvent is ethanol.

25. The method of claim 22, further comprising the step of encapsulating the powder into a capsule.

26. The method of claim 22, wherein the liposomal carrier complex is lecithin.

27. The method of claim 22, wherein the molecule of interest is a water-soluble vitamin.

28. The formulation of claim 1, further comprising an estrogen or progesterone.

29. The formulation of claim 1, wherein the liposomal carrier complex is lecithin.

30. The formulation of claim 3, wherein the treatment has a reduced risk of cardiovascular disease as compared to control treatments comprising the formulation without the flavonoid.
